(19) United States
(12) Patent Application Publication
(10) Pub. No.: US 2010/0137240 A1
(43) Pub. Date: Jun. 3, 2010

(54) USE OF NUCLEOSIDE DERIVATIVES AS ANTI-KOI HERPES VIRUS IN FISH

(75) Inventors: Berenice Costes, Liege (BE); Johan Neyts, Kessel-Lo (BE); Alain Vanderplasschen, Tubize (BE)

Correspondence Address: CLARK & ELBING LLP, 101 FEDERAL STREET, BOSTON, MA, 02110, US

(73) Assignees: Katholieke Universiteit Leuven, Leuven (BE); Universite de Liege, Liege (BE)

(21) Appl. No.: 12/600,110

(22) PCT Filed: May 16, 2008
(86) PCT No.: PCT/BE2008/000042

§ 371 Date: Nov. 13, 2009

(30) Foreign Application Priority Data
May 16, 2007 (GB) 0709408.9

(51) Int. Cl. A61K 31/7052; C07H 19/04; C07H 19/10; C07H 19/20; A61P 31/12

(52) U.S. Cl. 514/52; 536/26.1; 536/26.8; 536/26.7

(57) ABSTRACT

The present invention relates to the use of N-phosphonylmethoxyethyl nucleoside analogs for manufacturing a medicament for the treatment or prevention of Koi Herpes virus infections in fish, especially in carps.

### FIELD OF THE INVENTION

The present invention relates to the use of N-phosphonylmethoxyethyl nucleoside analogs for for the manufacture of a medicament for the treatment or prevention of Koi Herpes virus (KHV) infections in fish, especially in carps.

### BACKGROUND OF THE INVENTION

Common carp (Cyprinus carpio carpio) is a fish species that is widely cultivated for human food; 1.5 million metric tons is harvested annually, principally in China and many other Asian and European countries (www.fao.org). Unlike common carp, the koi subspecies (Cyprinus carpio koi) is a beautiful and colorful fish, and it has become part of a worldwide hobby consisting of keeping the fish in backyard ponds and large display aquaria for personal pleasure and competitive showing.

Koi herpesvirus (KHV) is an emerging virus that infects common carp and its varieties such as mirror, leather, koi and ghost carp. The disease is of global significance because of the extensive international trade in highly prized and expensive ornamental koi.

Koi carp is an important ornamental fish of very high added value in the fish farming industry in Asia. The practice of selectively breeding common carp for striking body colors, leading to a type called koi, has created a worldwide market for the ornamental fish hobby and competitive showing, where individual fish may exceed $100,000. In recent years, the industry has been suffering from a severe blow through the infection of Koi Herpes virus (KHV). The outbreak of KHV is found in some Asian countries, such as Taiwan, Hong Kong and Indonesia, as well as in Israel and Europe. The KHV disease process is highly contagious and is characterized by causing massive morbidity and mortality rate ranging between 80% and 90%. According to an

Indonesian research analysis, the process of KHV infection in East Java has affected over 5000 fish farmers and caused a loss of US$ 0.5 million from March to October 2002.

The KHV infection spread is furthermore of great importance because of the high economic importance of common carp as a food fish in countries such as China. Common carp also represents a significant resource in the United Kingdom as a major target species for freshwater anglers.

Following the initial identification of KHV in the late 1990's the virus has spread across the world. There is now a major concern over the potential spread of KHV into wild stocks of common carp which sustain carp fishing as an industry estimated to be worth over £1.5 billion pounds a year.

With the growth of angling and the demand for larger numbers of expensive, specimen carp, the number of stocking events (which generally occur during the colder months of the year to minimize transport stress) increases the risk of introducing unhealthy fish or fish harboring covert KHV infections. This creates a problem for the carp fisheries that may not be easily resolved. Screening tools based on detection of viral genome by PCR or on ELISA to detect specific antibodies for KHV produced by common carp after exposure to the virus are being used to detect fish carrying the infection and so prevent their introduction into fisheries.

Therapies for KHV-infections have not been developed up to date. It was found that temperature is the predetermining factor that controls whether KHV develops into a lethal infection and therefore, it has been advised to control water temperature. Acyclovir has already been tested for the treatment of KHV-infections, but this anti-HSV (Herpes simplex virus) drug widely used in humans is inactive against KHV infections, as demonstrated and explained by Ilouze et al. in FEBS Letters (2006) 580(18) 4473-4478. Acyclovir is an antiviral drug for the treatment of herpes virus infections in man and is highly effective against various human herpes viruses, including herpes simplex virus type 1 and type 2. Many compounds that exhibit selective antiviral activity against herpes viruses that cause disease in man are known and in clinical use. These include, besides acyclovir (ACV) and its oral prodrug form valacyclovir (valACV), ganciclovir (GCV) and its oral prodrug form valganciclovir (valGCV), brivudin (BVDV), foscarnet (PFA), cidofovir (HPMPC).

An alternative therapy or a therapy with (high) activity for KHV infections in fish, especially carps, would be of high economic importance in order to decrease the loss of fish due to high mortality. Therefore, there is a clear need in the art for therapeutic or preventive (prophylactic) methods for KHV-infections in fish. One goal of the present invention is therefore to provide the use of a class of compounds as anti-Koi Herpes virus active agents for manufacturing medicaments for treating or preventing KHV-infections in fish, especially in carps.

### SUMMARY OF THE INVENTION

It was surprisingly found that N-phosphonylmethoxyethyl nucleoside analogs, in particular N-2-phosphonylmethoxyethyl nucleoside analogs such as described in EP-253,412-B, more in particular PMEDAP (N-phosphonylmethoxyethyl-2,6-diaminopurin-9-yl), are highly active agents against KHV-infections in fish while it is shown that other compounds known as anti-herpes agents in mammals and man, such as acyclovir, brivudin, foscamet and ganciclovir, are very weakly active or inactive agents against KHV-infections in fish.

The present invention thus provides the use of N-phosphonyl-methoxyethyl nucleoside analogs, in particular N-2-phosphonyl-methoxyethyl nucleoside analogs such as described in EP-253,412-B, more in particular PMEDAP (N-phosphonylmethoxyethyl-2,6-diaminopurin-9-yl), pro-drugs, pharmaceutically or veterinary acceptable salts, tautomers, isomers and solvates thereof, for the manufacture of a medicament for the prevention or treatment of KHV-infections in fish, especially in carps. The present invention also relates to N-phosphonylmethoxyethyl nucleoside analogs, a pro-drug or solvate thereof for use as a medicament for the prevention or treatment of KHV-infections. The present invention also provides veterinary compositions comprising a KHV inhibiting amount of a N-phosphonyl-methoxyethyl nucleoside analog, in particular a N-2-phosphonylmethoxyethyl nucleoside analog such as described in EP-253,412-B, more in particular PMEDAP (N-phosphonylmethoxyethyl-2,6-diaminopurin-9-yl), a pro-drug, a pharmaceutically or veterinary acceptable salt, tautomer, isomer or solvate thereof having a therapeutic or preventive activity on KHV-infections in fish, optionally in combination with one or more suitable veterinary excipients, and the use thereof for treating or preventing KHV-infections in fish, especially in carps. The present invention furthermore provides a method of treating or preventing KHV-infections in a fish, comprising administering to the fish in need of such treatment a therapeutically effective amount or a KHV-inhibiting amount of such a N-phosphonylmethoxyethyl nucleoside analog, pro-drug or solvate thereof. The invention also provides for the use of N-phosphonylmethoxyethyl nucleoside analogs as inhibitors of KHV, for the prevention or treatment of KHV-infections, especially in the form of a combination with one or more other agents being biologically active against KHV in fish. The present invention also provides for the in vitro use of such a N-phosphonylmethoxyethyl nucleoside analog, a pro-drug or a solvate thereof as anti-Koi Herpes virus active compound.

Multiple N-phosphonylmethoxyethyl nucleoside analogs referred to in the different aspects of the present invention are already known in the art and include N-phosphonylmethoxyethyl derivatives of purine or pyrimidine bases, or analogs of these bases known in the art, as well as pharmaceutically acceptable salts, tautomers or isomers thereof.

In a particular embodiment of the present invention, the N-phosphonylmethoxyethyl derivatives of purine or pyrimidine bases, or analogs thereof, include compounds according to the structural formula (I) as already described in EP-253,412-B,

B—CH₂—CH₂—O—CH₂—P(═O)(OH)₂ (I)

wherein B is an optionally substituted pyrimidin-1-yl, pyrimidin-3-yl, purin-3-yl, purin-7-yl or purin-9-yl residue, or an aza, deaza, deoxy or deamino analogue thereof, and the salts thereof with alkali, metals, ammonia or amines.

In another particular embodiment of the present invention, the B residue may be the aza, deaza, deoxy or deamino analogues of uracil, thymine, cytosine, guanine, adenine, hypoxanthine or xanthine.

In another particular embodiment of the present invention, the residue B is 6-alkylpurin-3-yl, 6-alkylpurin-7-yl or 6-alkylpurin-9-yl or a derivative thereof.

In yet another particular embodiment of the present invention, the B residue is substituted with a methyl, amino, bromo, fluoro, hydroxy and/or a thio group.

In yet another particular embodiment of the present invention, B may be a moiety selected from the group consisting of pyrimidin-1-yl, pyrimidin-3-yl, purin-3-yl, purin-7-yl and purin-9-yl, which moiety may be unsubstituted or substituted with 1, 2 or 3 substituents independently selected from the group consisting of methyl, amino, cyclopropylamino, bromo, fluoro, hydroxyl and thio.

In yet another particular embodiment of the present invention, B may be a moiety selected from the group consisting of aza, deaza, deoxy or deamino analogues of uracil, thymine, cytosine, guanine, adenine, hypoxanthine and xanthine, which moiety may be unsubstituted or substituted with 1, 2 or 3 substituents independently selected from the group consisting of methyl, amino, bromo, fluoro, hydroxyl and thio.

In still another embodiment of the present invention, the residue B may be selected from the group consisting of uracil-1-yl, Thymin-1-yl, Cytosin-1-yl, 6-Methylpurin-9-yl, Guanin-9-yl, Hypoxanthin-9-yl, Adenin-9-yl, 2-aminoadenin-9-yl (2,6-diaminopurin-9-yl), 8-Bromoadenin-9-yl, 2-Aminopurin-9-yl, 6-Hydrazinopurin-9-yl, 7-Deaza-8-azaadenin-9-yl, 7-Deaza-8-azahypoxanthin-9-yl, 5-Methylcytosin-1-yl, 5-Fluorouracil-1-yl, Guanin-7-yl, Adenin-3-yl, Hypoxanthin-9-yl, 2-Methyladenin-9-yl, 2-Methylthioadenin-9-yl, N⁶-Dimethyladenin-9-yl, 8-Hydroxyadenin-9-yl, 6-Hydroxylaminopurin-9-yl, 6-Thiopurin-9-yl, Purin-9-yl and Xanthin-9-yl.

In yet another particular embodiment of the present invention, B may be adenin-9-yl which is unsubstituted or substituted with 1, 2 or 3 substituents independently selected from the group consisting of methyl, amino, bromo, fluoro, hydroxyl and thio.

In a more particular embodiment of the present invention, B is 2-aminoadenin-9-yl (2,6-diaminopurin-9-yl).

In another particular embodiment, the N-phosphonylmethoxyethyl nucleoside analogs of the invention are selected from PMEDAP (9-(2-phosphonylmethoxyethyl)-2,6-diaminopurine) and PMEA (9-[2-phosphonylmethoxyethyl]adenine).

In yet another particular embodiment of the present invention, the N-phosphonylmethoxyethyl derivatives of purine or pyrimidine bases, or analogs thereof, include compounds according to the following literature references:
Hatse et al. in Biochemical Pharmacology (1999) 58(2):311-323, i.e. N⁶-cyclopropyl-PMEDAP a derivative of 9-(2-phosphonylmethoxyethyl)-2,6-diaminopurine), and
Horejsi et al. in Bioorganic and Medicinal Chemistry (2006) 14(23) 8057-8065, i.e. tricyclic etheno analogs of PMEDAP and PMEG such as, but not limited to, 9-substituted (2-(3H-imidazo[1,2-a]purin-3-yl)ethoxy)methylphosphonic and 4-substituted (2-(1H-imidazo[2,1-b]purin-1-yl)ethoxy)methylphosphonic acids including Diisopropyl (2-(2-amino-6-(cyclopropylamino)-9H-purin-9-yl)ethoxy)methylphosphonate (5a), Diisopropyl (2-(2-amino-6-(diethylamino)-9H-purin-9-yl)ethoxy)methylphosphonate (5b), Diisopropyl (2-(2-amino-6-(dimethylamino)-9H-purin-9-yl)ethoxy)methylphosphonate (5c), Diisopropyl (2-(2-amino-6-(pyrrolidin-1-yl)-9H-purin-9-ypethoxy)methylphosphonate (5d), Diisopropyl (2-(9-oxo-5,9-dihydro-3H-imidazo[1,2-a]purin-3-yl)ethoxy)methylphosphonate (7), Diisopropyl (2-(9-(cyclopropylimino)-5,9-dihydro-3H-imidazo[1,2-a]purin-3-yl)ethoxy)methylphosphonate (9a), Diisopropyl (2-(9-(dimethylamino)-3H-imidazo[1,2-a]purin-3-yl)ethoxy)methylphosphonate (9c) and diisopropyl (2-(4-(dimethylamino)-1H-imidazo[2,1-b]purin-1-yl)ethoxy)methylphosphonate (10c), Diisopropyl (2-(9-(pyrrolidin-1-yl)-3H-imidazo[1,2-a]purin-3-yl)ethoxy)methylphosphonate (9d) and Diisopropyl (2-(4-(pyrrolidin-1-yl)-1H-imidazo[2,1-b]purin-1-yl)ethoxy)methylphosphonate (10d), Diisopropyl (2-(9-amino-3H-imidazo[1,2-a]purin-3-yl)ethoxy)methoxyphosphonate (9e) and Diisopropyl (2-(4-amino-1H-imidazo[2,1-b]purin-1-yl)ethoxy)methylphosphonate (10e), Diisopropyl (2-(4-(diethylamino)-1H-imidazo[2,1-b]purin-1-ypethoxy)methylphosphonate (10b), Sodium (249-oxo-5,9-dihydro-3H-imidazo[1,2-a]purin-3-ypethoxy)methylphosphonate (8), (2-(9-(Cyclopropylimino)-5,9-dihydro-3H-imidazo[1,2-a]purin-3-yl)ethoxy)methylphosphonic acid (11a), (2-(9-(Dimethylamino)-3H-imidazo[1,2-a]purin-3-yl)ethoxy)methylphosphonic acid (11c), (2-(9-(Pyrrolidin-1-yl)-3H-imidazo[1,2-a]purin-3-yl)ethoxy)methylphosphonic acid (11d), (2-(9-Amino-3H-imidazo[1,2-a]purin-3-yl)ethoxy)methylphosphonic acid (11e), (2-(4-(Diethylamino)-1-H-imidazo[2,1-b]purin-1-yl)ethoxy)methylphosphonic acid (12b), (2-(4-(Dimethylamino)-1-H-imidazo[2,1-b]purin-1-ypethoxy)methylphosphonic acid (12c), (2-(4-(Pyrrolidin-1-yl)-1-H-imidazo[2,1-b]purin-1-ypethoxy)methylphosphonic acid (12d) and (2-(4-Amino-1H-imidazo[2,1-b]purin-1-yl)ethoxy)methylphosphonic acid (12e)

The kinds of fish that can be treated by any one of the above-mentioned N-phosphonylmethoxyethyl nucleoside analogs of the present invention include, but are not limited to, carps, more particularly carps selected from the common carp Cyprinus carpio carpio and its varieties such as mirror, leather, koi (Cyprinus carpio koi) and ghost carp. In a particular embodiment, the N-phosphonylmethoxyethyl nucleoside analogs of the present invention are used to treat or prevent KHV-infections in Koi carps.

Thus, a first aspect of the invention relates to the use of N-phosphonyl-methoxyethyl nucleoside analogs as described in the different embodiments herein above, a pro-drug or solvate thereof for the manufacture of a medicament or pharmaceutical composition for the prevention, improvement and/or treatment of a KHV-infection in fish, more in particular carps, yet more in particular Koi. This aspect of the invention relates to the N-phosphonylmethoxyethyl nucleoside analogs as described in the different embodiments herein above, for the treatment, improvement or prevention of KHV-infections in fish.

Another aspect of the present invention relates to a method of prevention, improvement or treatment of a KHV-infection in a fish, especially a carp, comprising administering to the fish in need of such treatment or prophylaxis a therapeutically or prophylactic effective amount, preferably a

KHV inhibiting amount, of a N-phosphonylmethoxyethyl nucleoside analog as described in any of the different embodiments herein above, a pro-drug, a pharmaceutically or veterinary acceptable salt, a tautomer, an isomer or a solvate thereof, as an active ingredient, preferably in admixture with at least a pharmaceutically or veterinary acceptable carrier.

The present invention also relates to the use of N-phosphonyl-methoxyethyl nucleoside analogs as described in any of the different embodiments herein above, a pro-drug, a pharmaceutically or veterinary acceptable salt, a tautomer, an isomer or a solvate thereof, in a combination preparation, e.g. a kit, together with one or more other anti-KHV compounds. Therefore, the present invention also relates to pharmaceutical or veterinary compositions or preparations comprising a N-phosphonylmethoxyethyl nucleoside analog as described in any of the different embodiments herein above, and another anti-KHV compound, preferably in the form of synergistic veterinary compositions or preparations.

### BRIEF DESCRIPTION OF THE DRAWINGS

FIG. 1 shows the effect of a pulse chase treatment with PMEDAP on KHV growth in vitro.

FIG. 2 shows ose-response curves representing the activity of PMEDAP to reduce the number and size of KHV-induced plaques in CCB cells. Data are presented as the mean value of three independent experiments together with standard deviation.

### DETAILED DESCRIPTION OF THE INVENTION

We herein show that selective anti-HSV molecules, such as acyclovir, HPMPC (cidofovir), BVDU (brivudin) and ganciclovir (GCV) have no effect or a very limited effect on KHV replication. The 50% effective concentration for inhibition of KHV replication of these molecules is 100 to 100.000 fold higher (thus less effective) than their IC50 values for in vitro inhibiting the replication of other herpes viruses (e.g. herpes simplex virus). However, according to the present invention, we surprisingly show that N-phosphonylmethoxyethyl nucleoside analogs such as defined or listed in the Summary of the Invention, are significantly much more efficient in inhibiting KHV replication in vitro than drugs such as acyclovir, ganciclovir, cidofovir and brivudin. In fact the N-phosphonylmethoxyethyl nucleoside analogs such as defined hereinabove are the most potent anti-KHV compounds among known antiviral compounds. More in particular, PMEDAP has been found to be highly active against KHV.

In view of the in vitro activity as anti-Koi Herpes virus compounds, such compounds are very useful for decreasing or inhibiting KHV-infections in cells in vitro, for example in cell culture.

According to one embodiment, compounds of the present invention with a high potency against KHV infections have a structure in accordance with formula (I),

B—CH₂—CH₂—O—CH₂—P(═O)(OH)₂ (I)

wherein B is an optionally substituted pyrimidin-1-yl, pyrimidin-3-yl, purin-3-yl, purin-7-yl or purin-9-yl residue, or an aza, deaza, deoxy or deamino analogue thereof, including salts thereof with alkali, metals, ammonia or amines.

In a particular embodiment of the present invention, the B residues are aza, deaza, deoxy or deamino analogues of uracil, thymine, cytosine, guanine, adenine, hypoxanthine or xanthine.

In another particular embodiment of the present invention, the residue B is 6-alkylpurin-3-yl, 6-alkylpurin-7-yl or 6-alkyl-9-yl or a derivative thereof.

In yet another particular embodiment of the present invention, the B residue is substituted a methyl, amino, bromo, fluoro, hydroxy and/or a thio group.

In yet another particular embodiment of the present invention, B is a moiety selected from the group consisting of pyrimidin-1-yl, pyrimidin-3-yl, purin-3-yl, purin-7-yl and purin-9-yl which is unsubstituted or substituted with 1, 2 or 3 substituents selected from the group consisting of methyl, amino, bromo, fluoro, hydroxyl and thio.

In yet another particular embodiment of the present invention, B is a moiety selected from the group consisting of aza, deaza, deoxy or deamino analog of uracil, thymine, cytosine, guanine, adenine, hypoxanthine or xanthine which is unsubstituted or substituted with 1, 2 or 3 substituents selected from the group consisting of methyl, amino, bromo, fluoro, hydroxyl and thio.

In still another more particular embodiment of the present invention, the residue B is selected from Uracil-1-yl, Thymin-1-yl, Cytosin-1-yl, 6-Methylpurin-9-yl, Guanin-9-yl, Hypoxanthin-9-yl, Adenin-9-yl, 2-Aminoadenin-9-yl, 8-Bromoadenin-9-yl, 2-Aminopurin-9-yl, 6-Hydrazinopurin-9-yl, 7-Deaza-8-azaadenin-9-yl, 7-Deaza-8-azahypoxanthin-9-yl, 5-Methylcytosin-1-yl, 5-Fluorouracil-1-yl, Guanin-7-yl, Adenin-3-yl, Hypoxanthin-9-yl, 2-Methyladenin-9-yl, 2-Methylthioadenin-9-yl, N⁶-Dimethyladenin-9-yl, 8-Hydroxyadenin-9-yl, 6-Hydroxylaminopurin-9-yl, 6-Thiopurin-9-yl, Purin-9-yl, or Xanthin-9-yl.

In yet another particular embodiment of the present invention, B is adenin-9-yl which is optionally substituted with 1, 2 or 3 substituents selected from the group consisting of methyl, amino, bromo, fluoro, hydroxyl and thio.

In a more particular embodiment of the present invention, B is 2-aminoadenin-9-yl (i.e. the compound is PMEDAP).

In yet another particular embodiment of the present invention, B is a pyrimidine or purine base, or an analogue thereof (as defined herein), optionally substituted with one or two substituents independently selected from the group consisting of halogen, hydroxyl, sulfhydryl, methyl, ethyl, isopropyl, amino, methylamino, ethylamino, trifluoromethyl and cyano.

The term “pyrimidine and purine bases” as used herein includes, but is not limited to, adenine, thymine, cytosine, uracyl, guanine and 2,6-diaminopurine and analogues thereof. A purine or pyrimidine base as used herein includes a purine or pyrimidine base found in naturally occurring nucleosides as mentioned above. An analogue thereof is a base which mimics such naturally occurring bases in such a way that their structures (the kinds of atoms and their arrangement) are similar to the naturally occurring bases but may either possess additional or lack certain of the functional properties of the naturally occurring bases. Such analogues include those derived by replacement of a CH moiety by a nitrogen atom (e.g. 5-azapyrimidines such as 5-azacytosine) or vice versa (e.g., 7-deazapurines, such as 7-deazadenine or 7-deazaguanine) or both (e.g., 7-deaza, 8-azapurines). By derivatives of such bases or analogues are meant those bases wherein ring substituents are either incorporated, removed, or modified by conventional substituents known in the art, e.g. halogen, hydroxyl, amino, (C₁-C₆)alkyl and others. Such purine or pyrimidine bases, and analogues thereof, are well known to those skilled in the art, e.g. as shown at pages 20-38 of WO 03/093290.

In yet another particular embodiment of the present invention, B may be selected from the group comprising pyrimidine bases represented by the structural formula (B):

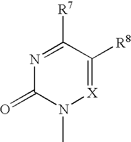

and purine bases represented by the structural formula (D):

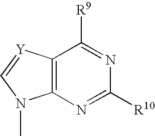

wherein:
R⁷ and R⁹ are independently selected from the group consisting of H, —OH, —SH, —NH₂, and —NH-Me;
R⁸ and R¹⁰ are independently selected from the group consisting of H, methyl, ethyl, isopropyl, hydroxyl, amino, ethylamino, trifluoromethyl, cyano and halogen; and
X and Y are independently selected from CH and N.

Just as a few non-limiting examples of pyrimidine analogues, can be named substituted uracils with the formula (B) wherein X is CH, R⁷ is hydroxyl, and R⁸ is methyl, ethyl, isopropyl, amino, ethylamino, trifluoromethyl, cyano, fluoro, chloro, bromo and iodo.

The term “C₁-C₆ alkyl” as used herein refers to normal, secondary, or tertiary hydrocarbon chains having from 1 to 6 carbon atoms. Examples thereof are methyl, ethyl, 1-propyl, 2-propyl, 1-butyl, 2-methyl-1-propyl (i-Bu), 2-butyl (s-Bu) 2-methyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-pentyl, and n-hexyl.

As used herein and unless otherwise stated, the term “cycloalkyl” means a monocyclic saturated hydrocarbon monovalent radical having from 3 to 6 carbon atoms, such as for instance cyclopropyl, cyclobutyl, cyclopentyl, cyclohexyl.

The term “(C₁-C₆) alkoxy” as used herein includes but is not limited to methoxy, ethoxy, propoxy, isopropoxy, butoxy, iso-butoxy, sec-butoxy, pentoxy, 3-pentoxy, or hexyloxy.

As used herein and unless otherwise stated, the term halogen means any atom selected from the group consisting of fluorine (F), chlorine (Cl), bromine (Br) and iodine (I).

The present invention also provides for the N-phosphonylmethoxyethyl nucleoside analogs as described herein for the prevention or treatment of KHV-infections in fish, more in particular in carps. The invention thus provides for the use of N-phosphonylmethoxyethyl nucleoside analogs as described herein for the manufacture of a medicament for the prevention or treatment of KHV-infections in fish, more in particular in carps.

The present invention furthermore provides for a KHV-inhibiting veterinary composition comprising a therapeutically effective amount of a N-phosphonylmethoxyethyl nucleoside analogs as described herein. The present invention provides furthermore for a veterinary composition comprising a N-phosphonylmethoxyethyl nucleoside analog, to be used for the prevention or treatment of a KHV-infection in fish, especially in carps.

The present invention also provides a method of prevention or treatment of KHV-infections in fish, especially in carps, by administering the said N-phosphonylmethoxyethyl nucleoside analogs to fish in need of such treatment or prophylaxis.

It will also be appreciated that the N-phosphonylmethoxyethyl nucleoside analogs and pharmaceutically or veterinary acceptable compositions of the present invention can be employed in combination therapies, that is, the compounds and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an N-phosphonyl-methoxyethyl nucleoside analogs may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”.

As an example, the N-phosphonylmethoxyethyl nucleoside analogs may be combined with antiviral compounds acting on other viral or non-viral targets, or with one or more interferon inducers such as, but not limited to, poly-IC (commercially available from AmpliGen), or interferon itself.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The N-phosphonylmethoxyethyl nucleoside analogs described herein are useful for the treatment or prophylaxis of KHV-infections in fish, especially in carps. When using N-phosphonylmethoxyethyl nucleoside analogs:
the N-phosphonylmethoxyethyl nucleoside analogs may be administered to the fish to be treated by any means well known in the art, i.e. orally, intraperitonealy, subcutaneously, intramuscularly, intradermally, intravenously, intraarterially, parenterally or by catheterization; the preferred routes of administration for the KHV-infected fish (e.g. a carp) is the intramuscular, intraperitoneal or subcutaneous route whereby a sterile solution of the compounds of the invention are injected; the most preferred route of administration is the intraperitoneal route whereby a sterile solution of a compound of the invention in a suitable liquid medium is injected to fish according to injection methods known in the art; and/or
   the therapeutically effective amount of the N-phosphonylmethoxyethyl nucleoside analog in fish is a KHV-inhibiting amount. This can be achieved by administration of the required dosage to obtain such plasma levels, the said effective amount may be divided into several sub-units per day or may be administered at more than one day intervals. The N-phosphonylmethoxyethyl nucleoside analogs of the present invention are preferably administered in a daily dosage ranging from about 10 mg/kg bodyweight to about 100 mg/kg bodyweight, preferably in a daily dosage of about 20 mg/kg bodyweight to about 60 mg/kg bodyweight. The daily dosage as well as the duration of administration may be varied, according to the common knowledge of the skilled person, depending upon parameters such as severity of the KHV infection, whether fish already shows infection symptoms or is treated prophylactically, etc.

More generally, the use of the N-phosphonylmethoxyethyl nucleoside analogs may also be in the diagnostic field and furthermore, any of the uses mentioned with respect to the present invention may be restricted to a non-medical use, a non-therapeutic use, a non-diagnostic use, or exclusively an in vitro use.

The N-phosphonylmethoxyethyl nucleoside analogs, prodrugs or solvates of the present invention as described herein also comprise pharmaceutically or veterinary acceptable salts, tautomers or isomers of the compounds of formula I as described hereunder.

The term “pharmaceutically acceptable salt” or “veterinary acceptable salt” as used herein refers to the therapeutically active non-toxic acid addition salt forms which the compounds of formula (I) may be able to form and which may conveniently be obtained by treating the base form of such compounds with an appropriate acid. Examples of such appropriate acids include, for instance, inorganic acids such as hydrohalic acids, e.g. hydrochloric or hydrobromic acid, sulfuric acid, nitric acid, phosphoric acid and the like; or organic acids such as, for example, acetic, propanoic, hydroxyacetic, 2-hydroxypropanoic, 2-oxopropanoic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclohexanesulfamic, salicylic (i.e. 2-hydroxybenzoic), p-aminosalicylic and the like.

This term also includes the solvates which the compounds of formula (I) as well as their salts are able to form with suitable inorganic or organic solvents, such as for example hydrates, alcoholates, ethers and the like.

Also included within the scope of this invention are the salts of the parent compounds with one or more amino acids, especially the naturally-occurring amino acids found as protein components. The amino-acid typically is one bearing a side chain with a basic or acidic group, e.g., lysine, arginine or glutamic acid, or a neutral group such as glycine, serine, threonine, alanine, isoleucine, or leucine.

Further examples of veterinary acceptable salts of the compounds of the invention include salts derived from an appropriate base, such as an alkali metal (for example, sodium), an alkaline earth (for example, magnesium), ammonium and NX⁴⁺ (wherein X is C₁-C₄ alkyl). Physiologically acceptable salts of an hydrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound containing a hydroxy group include the anion of said compound in combination with a suitable cation such as Na⁺ and NX⁴⁺ (wherein X typically is independently selected from H or a C₁-C₄ alkyl group). However, salts of acids or bases which are not physiologically acceptable may also find use, for example, in the preparation or purification of a physiologically acceptable compound. All salts, whether or not derived form a physiologically acceptable acid or base, are within the scope of the present invention.

The term “isomer” as used herein refers to all possible isomeric forms, including tautomeric forms, which the compounds of formula (I) may possess. Unless otherwise stated, the chemical designation of compounds denotes the mixture of all possible stereochemically isomeric forms, said mixtures containing all diastereomers and enantiomers (since the compounds of formula (I) may have at least one chiral center) of the basic molecular structure. More particularly, stereogenic centers may have either the R- or S-configuration, and substituents may have either cis- or trans-configuration. Pure isomeric forms of the said compounds are defined as isomers substantially free of other enantiomeric or diastereomeric forms of the same basic molecular structure. In particular, the term “stereoisomerically pure” or “chirally pure” relates to compounds having a stereoisomeric excess of at least about 80% (i.e. at least 90% of one isomer and at most 10% of the other possible isomers), preferably at least 90%, more preferably at least 94% and most preferably at least 97%. The terms “enantiomerically pure” and “diastereomerically pure” should be understood in a similar way, having regard to the enantiomeric excess, respectively the diastereomeric excess, of the mixture in question. Consequently, if a mixture of enantiomers is obtained during any of the following preparation methods, it can be separated by liquid chromatography using a suitable chiral stationary phase. Suitable chiral stationary phases are, for example, polysaccharides, in particular cellulose or amylose derivatives. Commercially available polysaccharide based chiral stationary phases are ChiralCel™ CA, OA, OB, OC, OD, OF, OG, OJ and OK, and Chiralpak™ AD, AS, OP(+) and OT(+). Appropriate eluents or mobile phases for use in combination with said polysaccharide chiral stationary phases are hexane and the like, modified with an alcohol such as ethanol, isopropanol and the like.

The terms cis and trans are used herein in accordance with Chemical Abstracts nomenclature and refer to the position of the substituents on a ring moiety. The absolute stereochemical configuration of the compounds of formula (I) may easily be determined by those skilled in the art while using well-known methods such as, for example, X-ray diffraction. Those of skill in the art will also recognize that the N-phosphonylmethoxyethyl nucleoside analogs may exist in many different protonation states, depending on, among other things, the pH of their environment. While the structural formulae provided herein depict the compounds in only one of several possible protonation states, it will be understood that these structures are illustrative only, and that the invention is not limited to any particular protonation state, any and all protonated forms of the compounds are intended to fall within the scope of the invention.

The KHV inhibiting compounds of the present invention may be formulated with conventional pharmaceutically acceptable carriers and excipients, which may be selected in accordance with ordinary practice. Tablets may contain one or more excipients, glidants, fillers, binders and the like. Aqueous formulations may be prepared in sterile form and, when intended for delivery by another route than oral administration, are generally isotonic. Formulations optionally contain pharmaceutically acceptable excipients such as those set forth in the “Handbook of Pharmaceutical Excipients” (1986) and may include ascorbic acid or other antioxidants, chelating agents such as EDTA, carbohydrates such as dextrin, hydroxyalkylcellulose, hydroxyalkylmethylcellulose, stearic acid and the like.

The term “pharmaceutically acceptable carrier” or “veterinary acceptable carrier” as used herein refers to any material or substance with which the active ingredient may be formulated in order to facilitate its application or dissemination to the locus to be treated, for instance by dissolving,dispersing or diffusing the said composition, and/or to facilitate its storage, transport or handling without impairing its biological effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, suspensions, ointments, creams, tablets, pellets or powders.

Suitable pharmaceutical carriers for use in the said pharmaceutical compositions and their formulation are well known to those skilled in the art, and there is no particular restriction to their selection within the present invention. They may also include additives such as wetting agents, dispersing agents, stickers, adhesives, emulsifying agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to fish, especially to carps. The pharmaceutical compositions of the present invention may be prepared in any known manner, for instance by homogeneously mixing, coating and/or grinding the active ingredients, in a one-step or multi-steps procedure, with the selected carrier material and, where appropriate, the other additives such as surface-active agents may also be prepared by inicronisation, for instance in view to obtain them in the form of microspheres usually having a diameter of about 1 to 10 μm, namely for the manufacture of microcapsules for controlled or sustained release of the active ingredients.

Suitable surface-active agents, also known as emulgent or emulsifier, to be used in the pharmaceutical compositions of the present invention are non-ionic, cationic and/or anionic materials having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphtalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidyl-ethanolamine, phosphatidylserine, phosphatidylglycerine, lysolecithin, cardiolipin, dioctanylphosphatidyl-choline, dipalmitoylphoshatidylcholine and their mixtures in any proportions.

Suitable non-ionic surfactants include polyethoxylated and poly-propoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylene-diaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenolpolyethoxyethanol, castor oil polyglycolic ethers, polypropylene/polyethylene oxide adducts, tributylphenoxypolyethoxyethanol, polyethyleneglycol and octylphenoxy-polyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C8-C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch, 2nd ed. (Hanser Verlag, Vienna, 1981) and “Encyclopaedia of Surfactants, (Chemical Publishing Co., New York, 1981).

While it is possible for the active ingredients to be administered alone it is preferable to present them as pharmaceutical formulations. The formulations for veterinary use of the present invention, in particular for administration to fish, comprise at least one active ingredient, as above described, together with one or more veterinary acceptable carriers therefore and optionally other therapeutic ingredients. Such formulations include those suitable for oral or parenteral (including subcutaneous, intraperitoneal, intramuscular, intravenous, intradermal, intrathecal and epidural) administration to fish, especially to carps. The formulations may conveniently be presented in unit dosage form and may be prepared by any of the methods well known in the art of pharmacy.

Such methods include the step of bringing into association the active ingredient of the invention with the one or more veterinary acceptable carriers. The formulations may be prepared by uniformly and intimately bringing into association the active ingredient with liquid carriers or finely divided solid carriers or both, and then, if necessary, shaping the product resulting from that association.

Formulations of the present invention suitable for oral administration may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

The formulations are optionally applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc), preferably 0.2 to 15% w/w and more preferably 0.5 to 10% w/w. When formulated in an ointment, the active ingredient(s) may be employed with either a paraffinic or a water-miscible ointment base. Alternatively, the active ingredients may be formulated in a cream with an oil-in-water cream base. If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG400) and mixtures thereof. The topical formulations may desirably include a compound which enhances absorption or penetration of the active ingredient through the skin or other affected areas. Examples of such dermal penetration enhancers include dimethylsulfoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Optionally, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

The choice of suitable oils or fats for the formulation is based on achieving the desired cosmetic properties, since the solubility of the active compound in most oils likely to be used in pharmaceutical emulsion formulations is very low. Thus the cream should optionally be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Preferred unit dosage formulations are those containing a daily dose or unit daily sub-dose, as herein above recited, or an appropriate fraction thereof, of an active ingredient.

It should be understood that in addition to the ingredients particularly mentioned above the formulations of this invention may include other agents conventional in the veterinary art having regard to the type of formulation in question, for example those suitable for oral administration may include flavoring agents.

Compounds of the invention can be used to provide controlled release pharmaceutical formulations containing as active ingredient one or more compounds of the invention (“controlled release formulations”) in which the release of the active ingredient can be controlled and regulated to allow less frequency dosing or to improve the pharmacokinetic or toxicity profile of a given invention compound. Controlled release formulations adapted for oral administration in which discrete units comprising one or more compounds of the invention can be prepared according to conventional methods. Additional ingredients may be included in order to control the duration of action of the active ingredient in the composition. Control release compositions may thus be achieved by selecting appropriate polymer carriers such as for example polyesters, polyamino acids, polyvinyl pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethyl-cellulose, polymethylmethacrylate and the other above-described polymers. Such methods include colloid drug delivery systems like liposomes, microspheres, micro-emulsions, nanoparticles, nanocapsules and so on. Depending on the route of administration, the veterinary composition may require protective coatings.

Pharmaceutical forms suitable for injectionable use include sterile aqueous solutions or dispersions or suspensions and sterile powders for the extemporaneous preparation thereof. Typical liquid carriers for this purpose therefore include biocompatible aqueous buffers (e.g. DMEM, see below), ethanol, glycerol, propylene glycol, polyethylene glycol and the like and mixtures thereof.

Preferably the N-phosphonylmethoxyethyl nucleoside analogs of the present invention are administered to fish as an injectable sterile solution comprising a suitable liquid medium (e.g. DMEM), whereby the N-phosphonylmethoxyethyl nucleoside analog is present in a concentration ranging from about 10 mg/ml to about 80 mg/ml.

When several active ingredients are used in combination, they do not necessarily bring out their joint therapeutic effect directly at the same time into the fish to be treated, the corresponding composition may also be in the form of a medical kit or package containing the two ingredients in separate but adjacent repositories or compartments. In the latter context, each active ingredient may therefore be formulated in a way suitable for an administration route different from that of the other ingredient, e.g. one of them may be in the form of an oral or parenteral formulation whereas the other is in the form of an ampoule for intravenous injection. Another embodiment of this invention relates to various precursor or “pro-drug” forms of the compounds of the present invention. It may be desirable to formulate the compounds of the present invention in the form of a chemical species which itself is not significantly biologically-active, but which when delivered to the fish will undergo a chemical reaction catalyzed by the normal function of the body of the fish, inter alia, enzymes present in the stomach or in blood serum, said chemical reaction having the effect of releasing a compound as defined herein. The term “pro-drug” thus relates to these species which are converted in vivo into the active pharmaceutical ingredient.

The pro-drugs of the present invention can have any form suitable to the formulator, for example, esters are non-limiting common pro-drug forms. In the present case, however, the pro-drug may necessarily exist in a form wherein a covalent bond is cleaved by the action of an enzyme present at the target locus. For example, a C—C covalent bond may be selectively cleaved by one or more enzymes at said target locus and, therefore, a pro-drug in a form other than an easily hydrolysable precursor, inter alia an ester, an amide, and the like, may be used. The counterpart of the active pharmaceutical ingredient in the pro-drug can have different structures such as an amino acid or peptide structure, alkyl chains, sugar moieties and others as known in the art.

For the purpose of the present invention the term “therapeutically suitable pro-drug” is defined herein as “a compound modified in such a way as to be transformed in vivo to the therapeutically active form, whether by way of a single or by multiple biological transformations, when in contact with the tissues of the fish to which the pro-drug has been administered, and without undue toxicity, irritation, or allergic response, and achieving the intended therapeutic outcome”.

More specifically the term “prodrug”, as used herein, relates to an inactive or significantly less active derivative of a compound such as represented by the structural formula (I), which undergoes spontaneous or enzymatic transformation within the body in order to release the pharmacologically active form of the compound. For a comprehensive review, reference is made to Rautio J. et al. (“Prodrugs: design and clinical applications” Nature Reviews Drug Discovery, 2008, doi: 10.1038/nrd2468).

In particular for the purpose of the present invention, pro-drugs of compounds represented by structural formula (I) may be formed as follows: a free phosphate acid function is available for prodrug formation as described in detail by Hecker S. J. and Erion M. D. (“Prodrugs of phosphates and phosphonates” Journal of Medicinal Chemistry (2008) doi: 10.1021/jm701260b).

The N-phosphonylmethoxyethyl nucleoside analogs used in the veterinary treatment of this invention can be prepared as described in EP-0 253 412-B, or the two other literature references cited supra.

KHV inhibitory activity can be readily detected using the assays described herein, as well as other assays known to those of ordinary skill in the art. In general, carp cells are infected with KHV and are incubated with the compounds to be tested for inhibitory activity. A more detailed description is provided in the examples.

The following examples are provided for the purpose of illustrating the present invention and by no means should be interpreted to limit the scope of the present invention.

### Example 1

### Inhibition of Koi Herpes Virus Infections in Cell Culture

Compounds for testing KHV-antiviral activity were dissolved in dimethylsulfoxide (DMSO) at a concentration of 20 mg/ml, then filtrated through a 0.45 μm filter to obtain a sterile solution. Common carp brain (CCB) cells were grown in minimum essential medium (MEM, commercially available from Invitrogen) containing 4.5 g/l glucose (D-glucose monohydrate, Merck) and 10% fetal calf serum (FCS, commercially available from BioWhittaker). Cells were incubated at 25° C. in a humid atmosphere containing 5% CO₂. Confluent cell monolayers grown in 24 wells cluster dishes were inoculated with 200 plaque forming units (PFU) of KHV in 0.2 ml of serum free Dulbecco's Modified Eagle Medium (DMEM, commercially available from Invitrogen). After an incubation period of 2 hours, cells were overlaid with DMEM (4.5 g/l glucose, 10% FCS) containing increasing doses of the relevant compounds (3 μg/ml; 6.5 μg/ml; 12.5 μg/ml; 25 μg/ml; 50 μg/ml and 100 μg/ml respectively). Five days post infection (pi), viral plaques or virus induced cytopathic effects were observed by staining cells with a crystal violet (Merck) solution.

The results are summarized in table 1 showing the IC50s for the compounds tested. The compounds of the invention, PMEDAP and PMEA show good activity against KHV replication. PMEDAP resulted in complete inhibition of KHV replication at a concentration of 12.5 μg/ml; PMEA at a concentration of 25 μg/ml. None of the other known antiviral compounds was able to completely inhibit KHV replication in vitro.

TABLE 1

| | IC50 |
|---|---|
| Compound | (μg/ml) |
| PMEDAP | 4.8 |
| PMEA (adefovir) | 6.5 |
| BVDU (brivudin) | 100 |
| GCV (ganciclovir) | 100 |
| PFA (foscarnet) | >100 |
| HPMPC | 50 |

Alternatively, PMEDAP was dissolved in DMEM and propyleneglycol (33.3%)/ethanol(8.3%) (PE buffer) respectively, at a concentration of 10 mg/ml. Common carp brain (CCB) cells were grown in minimum essential medium (MEM, commercially available from Invitrogen) containing 4.5 g/l glucose (D-glucose monohydrate, Merck) and 10% fetal calf serum (FCS, commercially available from BioWhittaker). Cells were incubated at 25° C. in a humid atmosphere containing 5% CO₂. Confluent cell monolayers grown in 24 wells cluster dishes were inoculated with 200 plaque forming units (PFU) of KHV in 0.2 ml of serum free Dulbecco's modified eagle medium (DMEM, commercially available from Invitrogen). After an incubation period of 2 hours, cells were overlaid with DMEM (4.5 g/l glucose, 10% FCS) containing increasing doses of the compound (3 μg/ml; 6.5 μg/ml; 12.5 μg/ml; 25 μg/ml; 50 μg/ml; and 100 μg/ml respectively). Five days post infection (pi), viral plaques or virus induced cytopathic effects were observed by staining cells with a crystal violet (Merck) solution. Results are shown in FIG. 2.

### Example 2

### Effect of a Pulse Chase Treatment with PMEDAP on KHV Growth In Vitro

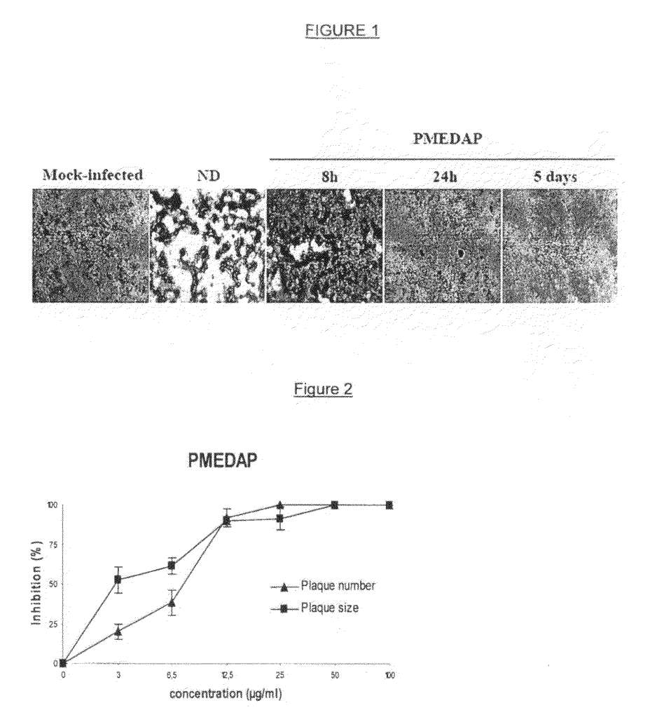

PMEDAP was dissolved in DMSO at a concentration of 20 mg/ml then filtrated through a 0.45 μm filter. CCB cells were grown in 24-well culture plates in MEM containing 4.5 g/l glucose and 10% FCS. Cells were inoculated with 200 PFU of KHV in 0.2 ml of serum free DMEM. After 2 hours incubation, cells were overlaid with DMEM (4.5 g/l glucose, 10% FCS) containing 50 μg/ml of PMEDAP. Mock-infected cells and untreated infected cells (ND) were included as negative controls. At successive intervals pi, cells were washed twice with PBS then overlaid with complete DMEM. Five days pi, viral plaques were observed by a crystal violet assay. The results are shown in FIG. 1. Incubation of infected cells with PMEDAP for only the first 24 hours post infection resulted in a complete inhibition of virus induced cytopathic effect observed 5 days post-infection.

### Example 3

### In Vivo Koi Carps Toxicity Experiment with PMEDAP

Naïve adult koi carps were kept in 150-liter tanks with filtrated water maintained at 23° C. One group (n=12) was treated for 10 consecutive days, by intraperitoneal injection of PMEDAP (50 mg/kg) dissolved in 0.2 ml of DMEM. The other group (n=12), used as negative control, was injected with 0.2 ml DMEM. After 10 days, all fish were examined and weighed. Three fishes of each group were euthanized. Liver and kidney explants were treated for anatomopathology analysis.

This experiment did not reveal any toxic effect of PMEDAP (see table 2). None of the fishes injected with PMEDAP died nor exhibited clinical signs. The mean weight of fishes infected with PMEDAP or DMEM were statistically similar.

TABLE 2

| | Mean initial | Mean final | Survival | Clinical | Microscopic |
|---|---|---|---|---|---|
| Carp | weight (g) | weight (g) | (%) | signs | lesions |
| Untreated | 62.8 | 66.2 | 100 | None | None |
| Treated | 66.1 | 71.5 | 100 | None | None |

An additional experiment was performed as following: Naive adult koi carps were kept in 60-liter tanks with filtrated water maintained at 23° C. Four groups (5 koi per group) were treated for 10 consecutive days, by intramuscular injection of 20 mg/kg PMEDAP ((“PMEDAP 20”) or 50 mg/kg PMEDAP (“PMEDAP 50”)) dissolved in 0.1 ml of DMEM. Two other groups (5 koi per group), used as negative controls, were injected with 0.1 ml of DMEM. After 10 days, all fish were examined and weighed. Three fishes of each group were euthanized. Liver and kidney explants were treated for anatomopathology analysis. The results are summarized in table 3.

| | Mean initial | Mean final | Survival | Clinical | Microscopic |
|---|---|---|---|---|---|
| Fish | weight (g) | weight (g) | (%) | signs | lesions |
| DMEM | 20.9 | 20.2 | 100 | None | None |
| PMEDAP 20 | 23.1 | 21.3 | 100 | None | None |
| PMEDAP 50 | 16.3 | 15.3 | 100 | None | None |

1. The use of a N-phosphonylmethoxyethyl nucleoside analog, a pro-drug, a salt or a solvate thereof, for manufacturing a medicament for the prevention or treatment of Koi Herpes virus infections in fish.

2. The use of claim 1, wherein said salt is a salt formed with an alkali, a metal, ammonia or an amine.

3. The use of claim 1 or claim 2, wherein said N-phosphonylmethoxyethyl nucleoside analog is a N-2-phosphonylmethoxyethyl nucleoside analog.

4. The use of any one of claims 1 to 3, wherein said N-phosphonyl-methoxyethyl nucleoside analog is according to the structural formula (I),
B—CH₂—CH₂—O—CH₂—P(═O)(OH)₂ (I)
wherein B is an optionally substituted pyrimidin-1-yl, pyrimidin-3-yl, purin-3-yl, purin-7-yl or purin-9-yl residue, or an aza, deaza, deoxy or deamino analogue thereof and the salts thereof

5. The use of claim 4, wherein B is an optionally substituted aza, deaza, deoxy or deamino analog of uracil, thymine, cytosine, guanine, adenine, hypoxanthine or xanthine.

6. The use of claim 4, wherein B is an optionally substituted adenin-9-yl moiety.

7. The use of claim 4, wherein B is 2-amino-adenin-9-yl.

8. The use of any one of claims 1 to 7, wherein the fish is a carp.

9. The use of any one of claims 1 to 8, wherein the fish is a Cyprinus carpio koi.

10. The use according to any one of claims 1 to 9, wherein said medicament is for administration via an intramuscular, intraperitoneal or subcutaneous route.

11. The use according to any one of claims 1 to 10, wherein said medicament is a sterile injectable solution of the N-phosphonylmethoxyethyl nucleoside analog, pro-drug or solvate thereof.
